(19) United States
(12) Patent Application Publication
(10) Pub. No.: US 2016/0317026 A1
(43) Pub. Date: Nov. 3, 2016

(54) OPTICAL COHERENCE TOMOGRAPHY SYSTEM FOR HEALTH CHARACTERIZATION OF AN EYE

(71) Applicant: UNIVERSITY OF SOUTHERN CALIFORNIA

(72) Inventors: Jeffrey P. Fingler, Orange, CA (US); Scott E. Fraser, Glendale, CA (US)

(73) Assignee: UNIVERSITY OF SOUTHERN CALIFORNIA, Los Angeles, CA (US)

(21) Appl. No.: 15/106,966

(22) PCT Filed: Mar. 4, 2015
(86) PCT No.: PCT/US15/18637

§ 371 Date: Jun. 21, 2016

Related U.S. Application Data
(60) Provisional application No. 61/947,856, filed on Mar. 4, 2014.
(60) Provisional application No. 62/112,537, filed on Feb. 5, 2015.

(51) Int. Cl. A61B 3/10; A61B 3/00; A61B 3/12

(52) U.S. Cl. CPC A61B 3/102; A61B 3/1241; A61B 3/1005; A61B 3/0025

(57) ABSTRACT

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to systems for health characterization of an eye by OCT angiography. This OCT angiography system may determine a feature of a vasculature within an eye tissue and thereby identify a vascular anomaly and a spatial location of the vascular anomaly within the eye tissue.

### CROSS-REFERENCE TO RELATED APPLICATIONS

This application is based upon and claims priority to U.S. provisional patent application 61/947,856, entitled “Retinal Microvascular Density Metric for Retinal Health Characterization,” filed Mar. 4, 2014, attorney docket number 028080-0990; and U.S. provisional patent application 62/112,537, entitled “Retinal Health Characterization by Optical Coherence Tomography,” filed Feb. 5, 2015, attorney docket number 064693-0314. The entire content of each of these provisional applications is incorporated herein by reference.

### STATEMENT REGARDING FEDERALLY SPONSORED RESEARCH

This invention was made with government support under Grant No. NIH STTR 1 R41 EY021054 awarded by National Institutes of Health (NIH). The government has certain rights in the invention.

### BACKGROUND

1. Technical Field

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to methods for health characterization of an eye by OCT angiography.

2. Description of Related Art

Optical coherence tomography (OCT) has become an important clinical imaging tool, since its introduction in 1991. For a background of OCT technology, see, for example, Drexler and Fujimoto et al. “Optical Coherence Technology: Technology and Applications” Springer, Heidelberg, Germany, 2008. This book is incorporated herein by reference in its entirety. OCT is based on an optical measurement technique known as low-coherence interferometry. OCT performs high resolution, cross-sectional imaging of internal microstructure of a physical object by directing a light beam to the physical object, and then measuring and analyzing magnitude and time delay of backscattered light.

A cross-sectional image is generated by performing multiple axial measurements of time delay (axial scans or A-scans) and scanning the incident optical beam transversely. This produces a two-dimensional data set of A-scans, which represents the optical backscattering in a cross-sectional plane through the physical object (i.e. B-scans). Three-dimensional, volumetric data sets can be generated by acquiring sequential cross-sectional images by scanning the incident optical beam in a raster pattern (three-dimensional OCT or 3D-OCT). This technique yields internal microstructural images of the physical objects with very fine details. For example, pathology of a tissue can effectively be imaged in situ and in real time with resolutions smaller than 15 micrometers.

Several types of OCT systems and methods have been developed, for example, Time-domain OCT (TD-OCT) and Fourier-domain OCT (FD-OCT). Use of FD-OCT enables high-resolution imaging of retinal morphology that is nearly comparable to histologic analysis. Examples of FD-OCT technologies include Spectral-domain OCT (SD-OCT) and Swept-source OCT (SS-OCT).

OCT may be used for identification of common retinovascular diseases, such as age-related macular degeneration (AMD), diabetic retinopathy (DR), and retinovascular occlusions. However, despite the rapid evolution of OCT imaging, current OCT technology may not provide adequate visualization of retinal and choroidal microvasculature. Thus, clinicians are often compelled to order both OCT and fluorescein angiography (FA) in patients with the retinovascular diseases.

Since their introduction more than 50 years ago, fluorescein angiography (FA) and indocyanine green angiography (ICGA) have been used for retinovascular imaging. This method typically involves an injection of fluorescent dye into the blood stream, and the perfusion of the dye into the retinal and choroidal blood vessels is observed optically on the fundus. An estimated 1 million FA studies are performed annually in the United States. Although FA has obvious value in revealing fine details of the microvasculature, it may require an intravenous injection and a skilled photographer and may be time-consuming. Minor side effects such as nausea, vomiting, and multiple needle sticks in patients with challenging venous access are not uncommon. Because fluorescein leaks readily through the fenestrations of the choriocapillaris, it may not be suitable for showing the anatomy of this important vascular layer that supplies the outer retina. ICGA provides improved visualization of choroidal anatomy because this dye is more extensively protein bound than fluorescein and may not leak into the extravascular space as readily. Furthermore, it may fluoresce at a longer wavelength than fluorescein and imaging can take place through pigment and thin layers of blood. Nevertheless, ICGA may fail to depict the fine anatomic structure of the choriocapillaris.

There has been increased interest in using data generated during FD-OCT imaging to generate angiographic images of the fundus. These angiograms can be implemented noninvasively without injection of fluorescent dye.

Recently, phase-variance OCT (PV-OCT) has been introduced to image retinal microvasculature. See, for example, Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; Fingler et al. “Mobility and transverse flow visualization using phase variance contrast with spectral domain optical coherence tomography” Opt. Express 2007; 15:12636-53; Fingler et al. “Phase-contrast OCT imaging of transverse flows in the mouse retina and choroid” Invest Ophthalmol. Vis. Sci. 2008;49:5055-9; Fingler et al. “Volumetric microvascular imaging of human retina using optical coherence tomography with a novel motion contrast technique” Opt. Express 2009;17:22190-200; Kim et al. “In vivo volumetric imaging of human retinal circulation with phase-variance optical coherence tomography” Biomed Opt Express [serial online] 2011; 2:1504-13; Kim et al. “Noninvasive imaging of the foveal avascular zone with high-speed, phase-variance optical coherence tomography” Invest. Ophthalmol. Vis. Sci. 2012; 53:85-92; and Kim et al. “Optical imaging of the chorioretinal vasculature in the living human eye” PNAS, August 27, 2013, vol. 110, no. 35, 14354-14359. All these publications and patent disclosures are incorporated herein by reference in their entirety.

PV-OCT uses software processing of data normally acquired, but not used, during FD-OCT imaging. With a different scanning protocol than found in commercial instruments, PV-OCT identifies regions of motion between consecutive B-scans that are contrasted with less mobile regions. In the retina and choroid, the regions with motion correspond to the vasculature; these vessels are readily differentiated from other retinal tissues that are relatively static.

An alternative method to acquire images of the retinal vasculature is Doppler OCT, which measures the change in scatterer position between successive depth scans and uses this information to calculate the flow component parallel to the imaging direction (called axial flow). Doppler OCT has been used to image large axial flow in the retina, but without dedicated scanning protocols this technique is limited in cases of slow flow or flow oriented transverse to the imaging direction. Because this technique depends on measuring motion changes between successive depth scans, as imaging speed improvements continue for FD-OCT systems, the scatterers have less time to move between measurements and the slowest motions become obscured by noise. This further reduces the visualization capabilities of typical Doppler OCT techniques.

In contrast, PV-OCT will be able to achieve the same time separations between phase measurements with increased FD-OCT imaging speeds, maintaining the demonstrated ability to visualize fast blood vessel and slow microvascular flow independently of vessel orientation.

Several groups in recent years have developed OCT imaging methods to push beyond conventional Doppler OCT imaging limitations. Some approaches involve increasing the flow contrast through hardware modifications of FD-OCT machines, such as in 2-beam scanning, or producing a heterodyne frequency for extracting flow components. Other investigators have used nonconventional scanning patterns or repeated B-scan acquisitions, such as used in PV-OCT to increase the time separation between phase measurements and enhance Doppler flow contrast of microvascular flow. In addition to phase-based contrast techniques to visualize vasculature, intensity-based visualization of microvasculature has been developed for OCT using segmentation, speckle-based temporal changes, decorrelation-based techniques, and contrast based on both phase and intensity changes. Each of these methods has varying capabilities in regard to microvascular visualization, noise levels, and artifacts while imaging retinal tissues undergoing typical motion during acquisition. Some of the noise and artifact limitations can be overcome with selective segmentation of the volumetric data or increased statistics through longer imaging times, but further analysis may be required to be able to compare all of the visualization capabilities from all these different systems.

For further description of OCT methods and systems, and their applications, for example, see: Schwartz et al. “Phase-Variance Optical Coherence Tomography: A Technique for Noninvasive Angiography” American Academy of Ophthalmology, Volume 121, Issue 1, January 2014, Pages 180-187; Sharma et al. “Data Acquisition Methods for Reduced Motion Artifacts and Applications in OCT Angiography” U.S. Pat. No. 8,857,988; Narasimha-Iyer et al. “Systems and Methods for Improved Acquisition of Ophthalmic Optical Coherence Tomography Data” U.S. Patent Application Publication No. 2014/0268046; Everett “Methods for Mapping Tissue With Optical Coherence Tomography Data” U.S. Pat. No. 7,768,652. All these publications and patent disclosures are incorporated herein in their entirety.

### SUMMARY

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to methods for health characterization of an eye by OCT angiography.

This disclosure relates to an optical coherence tomography (OCT) system for health characterization of an eye of a subject. The subject may be any mammal. The subject may be a human. This OCT system may have a configuration that (a) scans a tissue of the eye of a subject, which has a surface and a depth, with a beam of light that has a beam width and a direction; (b) acquires OCT signals from the scan; and (c) forms at least one B-scan cluster set using the acquired OCT signals.

Each B-scan cluster set may include at least two B-scan clusters. Each B-scan cluster may include at least two B-scans. Each B-scan may include at least two A-scans. Each B-scan cluster set may be parallel to one another and parallel to the direction of the beam of light. The B-scans within each B-scan cluster set may be parallel to one another and parallel to the direction of the beam of light.

The OCT system for health characterization of an eye may have a configuration to form more than one B-scan cluster set. Each B-scan cluster set may comprise any number of B-scan clusters. Each B-scan cluster may comprise any number of B-scans Each B-scan cluster may comprise any number of B-scans. Each B-scan may comprise any number of A-scans.

Each A-scan, each B-scan, each B-scan cluster, and each B-scan cluster set may be acquired over a period of time. That is each A-scan, each B-scan, each B-scan cluster, and each B-scan cluster set may be formed at a different time than all other A-scans, B-scans, B-scan clusters, and B-scan cluster sets, respectively. In this disclosure, “first formed” means first formed in time; “next formed” means next formed in time; and “last formed” means last formed in time.

Each A-scan may be separated from any next A-scan by a distance (“A-scan distance”). Each B-scan within each B-scan cluster may be separated from any next formed B-scan within that B-scan cluster by a distance (“intra-cluster distance”) in the range of 0 to half of the beam width. The last formed B-scan within each B-scan cluster may be separated from the first formed B-scan within any next formed B-scan cluster (“inter-cluster distance”) by at least one micrometer.

The OCT system for health characterization of an eye may have a configuration that calculates an OCT angiography data using the at least one B-scan cluster and motion occurring within the eye tissue. The OCT angiography data may be calculated by using variations of intensity and/or phase of the OCT signals. This calculation may provide contrast. These variations may be variations caused by flow, speckle, and/or decorrelation of the OCT signal caused by eye tissue motion and/or flow in blood vessels of the eye tissue.

This OCT system may have a configuration that measures a feature of a vasculature within the eye tissue by using the calculated angiography data. The feature of a vasculature may be any feature of the vasculature. For example, the feature of a vasculature may be a size of a blood vessel, a spatial distance between each blood vessel, a cross-sectional area of a blood vessel, number of blood vessels, a shape of a blood vessel, a volume of a blood vessel, and/or a spatial location of a blood vessel within the tissue. The size of a blood vessel may be any size of the blood vessel. For example, the size of a blood vessel may be its characteristic length, characteristic cross-sectional length, characteristic cross-sectional diameter, cross-sectional perimeter, or cross sectional circumference. The volume of blood vessels may be any volume of blood vessels. For example, the volume of blood vessels may be a total volume of blood vessels or a volume of blood vessels that have a specific blood vessel size (such as a volume distribution of the blood vessels based on the size of the blood vessels).

The measured feature of the vasculature may be used to calculate a blood vessel population. The blood vessel population may be any blood vessel population. For example, the blood vessel population may be (a) a size distribution of blood vessels, (b) a spatial distance distribution of the blood vessels; (c) a cross-sectional area distribution of blood vessels; (d) a spatial location distribution of blood vessels; (e) a number of blood vessels per cross-sectional unit area of the tissue, and/or volume of the tissue; (f) a total of cross-sectional area of blood vessels per unit area of the tissue; (g) a volume of blood vessels per unit volume of the tissue; or (h) combinations thereof. The calculated blood vessel population may be a number and/or a histogram.

The blood vessel population may be used to identify a vascular anomaly and a spatial location of the vascular anomaly within the eye tissue of the subject. For this identification, for example, the subject's blood vessel population may be compared with that of a healthy eye tissue. The healthy eye tissue may be a tissue of the other eye of the subject. The healthy eye tissue may be a tissue from another region of the same eye of the subject. The healthy eye tissue may be a tissue of an eye of another (e.g. healthy) subject. The blood vessel population of a healthy subject may be determined by acquiring OCT angiography signals from the healthy subject. A database of healthy objects' blood vessel population may be formed and used for this comparison purposes. The vascular anomaly and its spatial location within the subject's eye tissue may thereby be determined by comparing such blood vessel population calculations. For this identification, for example, the subject's blood vessel population information may be calculated more than once over a period of time. The vascular anomaly and its spatial location within the subject's eye tissue may be determined by comparing the blood vessel information calculated at a later time with that of an earlier time.

The OCT system may further have a configuration that acquires OCT signals to form at least two B-scan cluster sets. The spatial distance between last formed B-scan of any B-scan cluster of each B-scan cluster set and first formed B-scan of any B-scan cluster of next formed B-scan cluster set (“inter-cluster-set distance”) may be equal to or greater than 1 micrometer.

The OCT system for health characterization of an eye may also have a configuration that calculates a first OCT angiography data and a second OCT angiography data. The first OCT angiography data may be calculated at a first inter-cluster-set distance. And the second OCT angiography data may be calculated at a second inter-cluster-set distance. The second inter-cluster-set distance may be smaller than the first inter-cluster-set distance.

The first OCT angiography data may be used in identification of a vascular anomaly and a spatial location of the vascular anomaly within the eye tissue by using the blood vessel population. The second OCT angiography data may be calculated by scanning a tissue at or around the vascular anomaly and acquiring OCT signals from this scan. This system configuration may yield more detailed information related to the anomaly.

A non-transitory, tangible, computer-readable storage media containing a program of instructions that causes an optical coherence tomography (OCT) system for health characterization of an eye to run the program of instructions (“storage media”) is also within the scope of this disclosure.

The storage media may have a configuration that scans tissue of an eye of a subject, which has a surface and a depth, with a beam of light that has a beam width and a direction; acquires OCT signals from the scan; forms at least one B-scan cluster set using the acquired OCT signals; calculates OCT angiography data using the at least one B-scan cluster set formed and based on motion occurring within the eye tissue; and determines a feature of a vasculature within the eye tissue by using the calculated angiography data.

The storage media may have a further configuration that calculates a blood vessel population using the determined feature of the vasculature. The blood vessel population may be a size distribution of the blood vessels; a spatial distance distribution of the blood vessels; a cross-sectional area distribution of the blood vessels; a spatial location distribution of the blood vessels; a number of blood vessels per cross-sectional unit area of the tissue, or per volume of the tissue; a volume of blood vessels per volume of the tissue; a total cross-sectional area of blood vessels per unit area of the tissue; or a combination thereof.

The storage media may also have a further configuration that identifies a vascular anomaly and a spatial location of the vascular anomaly within the eye tissue by using the calculated blood vessel population.

The storage media may also have a further configuration that forms at least two B-scan cluster sets such that last formed B-scan of one of the B-scan clusters of each B-scan cluster set is separated from first formed B-scan of one of the B-scan clusters of the next formed B-scan cluster set (“inter-cluster-set distance”) by a first distance; calculates a first OCT angiography data using the at least two B-scan cluster sets and a motion occurring within the eye tissue; and identifies a vascular anomaly and a spatial location of the vascular anomaly within the eye tissue by using the blood vessel population. The storage media may also have a further configuration that forms at least two B-scan cluster sets at a second inter-cluster-set distance, at or around the spatial locations of the identified vascular anomaly such that the second inter-cluster-set distance is smaller than the first inter-cluster-set distance; and calculates a second OCT angiography data using the at least two B-scan cluster sets formed at the second inter-cluster-set distance and a motion occurring within the eye tissue. The storage media may also have a further configuration that determines a feature of blood vessels within the eye tissue by using the second OCT angiography data.

The storage media may also have a program of instructions that causes the computer system running the program of instructions to identify a vascular anomaly and spatial location of the vascular anomaly within the eye tissue by comparing the blood vessel population information with that of a healthy eye tissue.

The storage media may also have a program of instructions that causes the computer system running the program of instructions to identify a vascular anomaly and spatial location of the vascular anomaly within the eye tissue by calculating the blood vessel population information of the subject at different times and comparing the blood vessel population information calculated at a later time with that of an earlier time.

Any combination of above features, products and methods is within the scope of the instant disclosure.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates a generalized OCT system.

FIG. 2 schematically illustrates an example of a scanning configuration for the OCT system illustrated in FIG. 1.

FIG. 3 schematically illustrates a sagittal view of an exemplary left human eye.

FIG. 4 schematically illustrates cross sectional layers of an exemplary retina.

FIG. 5 shows a cross-sectional (2D) OCT image of the fovea region of an exemplary retina.

FIG. 6 shows (A) an exemplary en-face OCT angiography image of an exemplary retinal vasculature around optic disc, (B) a magnified region of the OCT image of (A).

FIG. 7 schematically illustrates visual field of a fundus of an exemplary left eye of a healthy human.

FIG. 8 shows an example of an intensity distribution of a beam of light, transverse to the propagation direction.

FIG. 9 schematically illustrates four B-scans, two B-scan clusters, and one B-scan cluster set by way of example that may be used for the calculation of an OCT angiography data.

FIG. 10 shows (A) an exemplary en-face OCT angiography image of retinal vasculature around optic disc of a healthy human, (B) an exemplary en-face OCT angiography image of retinal vasculature around optic disc of a human with diabetic retinopathy.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

This disclosure relates to the field of Optical Coherence Tomography (OCT). This disclosure particularly relates to methods and systems for providing larger field of view OCT images. This disclosure also particularly relates to methods and systems for OCT angiography. This disclosure further relates to methods for health characterization of an eye by OCT angiography.

This disclosure relates to an OCT system. The OCT system may comprise any interferometer that have optical designs, such as Michelson interferometer, Mach-Zehnder interferometer, Gires-Tournois interferometer, common-path based designs, or other interferometer architectures. The sample and reference arms in the interferometer may include any type of optics, for example bulk-optics, fiber-optics, hybrid bulk-optic systems, or the like.

The OCT system for health characterization of an eye or the OCT angiography system may also include any OCT system. Examples of the OCT systems may include Time-domain OCT (TD-OCT) and Fourier-domain, or Frequency-domain, OCT (FD-OCT). Examples of the FD-OCT may include Spectral-domain OCT (SD-OCT), Swept Source OCT (SS-OCT), and Optical frequency domain Imaging (OFDI).

The OCT system may use any OCT configuration that identifies and/or visualizes regions of motion (“OCT angiography”). The OCT angiography may use motion occurring within the physical object to identify and/or visualize regions with improved contrast based on variations in the intensity and/or phase of the OCT signal. For example, these variations are caused by flow, speckle or decorrelation of the OCT signal caused by eye motion or flow in blood vessels. For example, variation of OCT signals caused by blood flow in blood vessels may be used by OCT to identify and/or visualize retinal or choroidal vasculature in the eye through the OCT angiography. As a result, structures and functions can be visualized that cannot be identified through a typical OCT system. For example, choriocapillaris may become visible by using the OCT angiography.

Examples of the OCT angiography may include Phase Variance OCT (PV-OCT), Phase Contrast OCT (PC-OCT), Intensity/Speckle Variance OCT (IV-OCT), Doppler OCT (D-OCT), Power of Doppler Shift OCT (PDS-OCT), Split Spectrum Amplitude Decorrelation Analysis (SSADA), Optical Micro-angiography (OMAG), Correlation Mapping OCT (cmOCT), and the like.

Examples of the PV-OCT are disclosed by Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; Fingler et al. “Mobility and transverse flow visualization using phase variance contrast with spectral domain optical coherence tomography” Opt. Express [serial online] 2007; 15:12636-53; examples of the Speckle Variance OCT are disclosed by Mariampillai et al. “Speckle variance detection of microvasculature using swept-source optical coherence tomography,” Opt. Lett.33(13), 1530-1532 (2008); examples of the Correlation Mapping OCT method are disclosed by Enfield et al. “In vivo imaging of the microcirculation of the volar forearm using correlation mapping optical coherence tomography (cmOCT)” Biomed. Opt. Express 2,1184-1193 (2011); examples of the OMAG are disclosed by An et al. “In vivo volumetric imaging of vascular perfusion within human retina and choroids with optical micro-angiography” Opt. Express 16, 11438-11452 (2008); examples of the Power Doppler OCT are disclosed by Makita et al. “Optical coherence angiography” Opt. Express 14,7821-7840 (2006); examples of the SSADA are disclosed by Jia et al. “Split-spectrum amplitude-decorrelation angiography with optical coherence tomography,” Opt. Express20(4), 4710-4725 (2012). The entire contents of these disclosures are incorporated herein by reference.

The OCT system for health characterization of an eye may comprise a generalized OCT system. For example, the OCT system may comprise at least one light source that provides the beam of light; at least one retro-reflector; at least one optical fiber coupler or at least one free space coupler that guides the beam of light to the physical object and to the at least one retro-reflector, wherein the beam of light guided to the physical object forms at least one backscattered light beam, and wherein the beam of light guided to the at least one retro-reflector forms at least one reflected reference light beam; at least one scanning optic that scans the at least one light beam over the physical object; and at least one detector. The at least one detector may combine the at least one backscattered light beam and the at least one reflected light beam to form light interference, detect magnitude and time delay of the at least one backscattered light beam, and forms at least one OCT signal. The at least one optical fiber coupler or the at least one free space coupler may guide the at least one backscattered light beam and the at least one reflected light beam to the at least one detector. The OCT system may further comprise at least one processor that obtains and analyzes the at least one OCT signal formed by the at least one detector, and forms an image of the physical object. The OCT system may also further comprise at least one display that displays the image of the physical object.

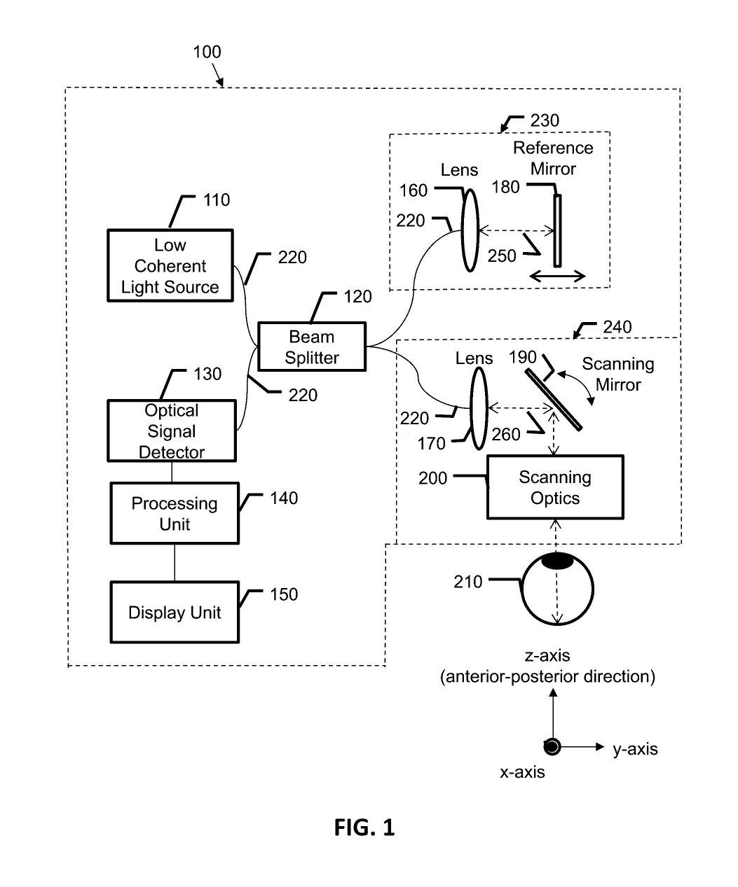

Examples of a generalized OCT system schematically shown in FIG. 1 are disclosed by Fingler et al. “Dynamic Motion Contrast and Transverse Flow Estimation Using Optical Coherence Tomography” U.S. Pat. No. 7,995,814; Fingler et al. “Dynamic Motion Contrast and Transverse Flow Estimation Using Optical Coherence Tomography” U.S. Pat. No. 8,369,594; and Sharma et al. in a U.S. Pat. No. 8,857,988, entitled “Data Acquisition Methods for Reduced Motion Artifacts and Applications in OCT Angiography”. These disclosures are incorporated herein by reference in their entirety. The OCT system may comprise this generalized OCT system.

The OCT system 100 may comprise at least one light source 110, at least one scanning optic 200, at least one retro-reflector 180, at least one optical fiber coupler 220 or at least one free space coupler, at least one detector 130, at least one processing unit 140, and at least one display unit 150. The OCT system may further comprise a scanning mirror 190.

The at least one light source 110 may comprise any light source, for example, a low coherent light source. Light from the light source 110 may be guided, typically by using at least one optical fiber coupler 220 to illuminate a physical object 210. An example of the physical object 210 may be any tissue in a human eye. For example, the tissue may be a retina. The light source 110 may be either a broadband low coherence light source with short temporal coherence length in the case of SD-OCT or a wavelength tunable laser source in the case of SS-OCT. The light may be scanned, typically with the scanning optic 200 between the output of the optical fiber coupler 220 and the physical object 210, so that a beam of light (dashed line) guided for the physical object 210 is scanned laterally (in x-axis and/or y-axis) over the area or volume to be imaged. The scanning optic 200 may comprise any optical element suitable for scanning. The scanning optic 200 may comprise at least one component. The at least one component of the scanning optic 200 may be an optical component. Light scattered from the physical object 210 may be collected, typically into the same optical fiber coupler 220 used to guide the light for the illumination of the physical object 210. (The physical object 210 is shown in FIG. 1 only to schematically demonstrate the physical object 210 in relation to the OCT system 100. The physical object 210 is not a component of the OCT system 100.)

The OCT system 100 may further comprise a beam splitter 120 to split and guide the light provided by the light source 110 to a reference arm 230 and a physical object arm 240. The OCT system may also further comprise a lens 160 placed between the beam splitter 120 and the retro-reflector 180. The OCT system may also further comprise another lens 170 placed between the beam splitter 120 and the scanning optic 200.

Reference light 250 derived from the same light source 110 may travel a separate path, in this case involving the optical fiber coupler 220 and the retro-reflector 180 with an adjustable optical delay. The retro-reflector 180 may comprise at least one component. The at least one component of the retro-reflector 180 may be an optical component, for example, a reference mirror. A transmissive reference path may also be used and the adjustable delay may be placed in the physical object arm 240 or the reference arm 230 of the OCT system 100.

Collected light 260 scattered from the physical object 210 may be combined with reference light 250, typically in the fiber coupler to form light interference in the detector 130. Although a single optical fiber port is shown going to the detector 130, various designs of interferometers may be used for balanced or unbalanced detection of the interference signal for SS-OCT or a spectrometer detector for SD-OCT.

The output from the detector 130 may be supplied to the processing unit 140. Results may be stored in the processing unit 140 or displayed on the display 150. The processing and storing functions may be localized within the OCT system or functions may be performed on an external processing unit to which the collected data is transferred. This external unit may be dedicated to data processing or perform other tasks that are quite general and not dedicated to the OCT system.

Light beam as used herein should be interpreted as any carefully directed light path. In time-domain systems, the reference arm 230 may need to have a tunable optical delay to generate interference. Balanced detection systems may typically be used in TD-OCT and SS-OCT systems, while spectrometers may be used at the detection port for SD-OCT systems.

The interference may cause the intensity of the interfered light to vary across the spectrum. The Fourier transform of the interference light may reveal the profile of scattering intensities at different path lengths, and therefore scattering as a function of depth (z-axis direction) in the physical object. See for example Leitgeb et al. “Ultrahigh resolution Fourier domain optical coherence tomography,” Optics Express 12(10):2156, 2004. The entire content of this publication is incorporated herein by reference.

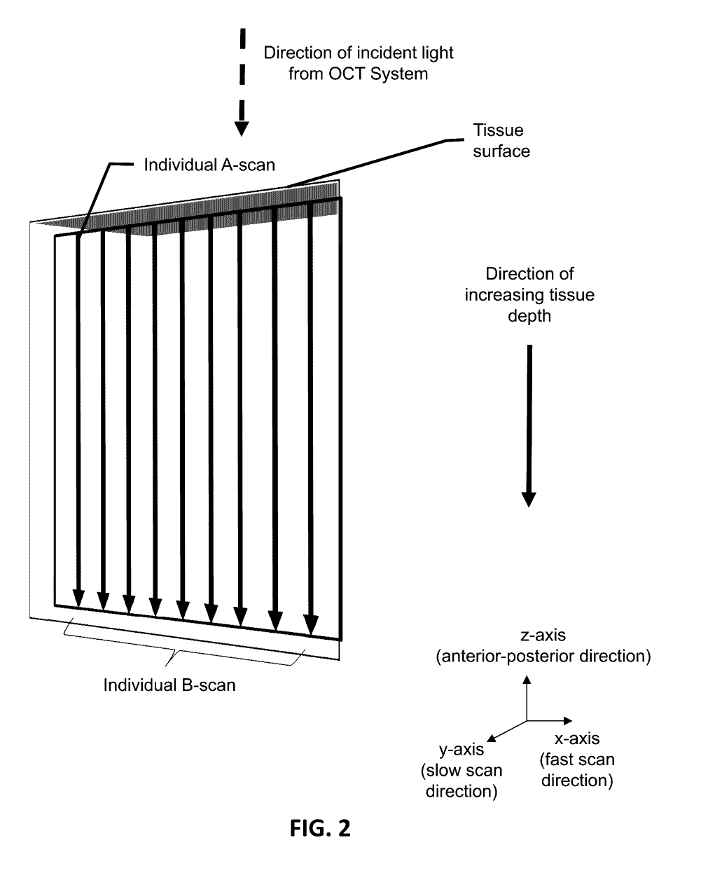

The profile of scattering as a function of depth is called an axial scan (A-scan), as schematically shown in FIG. 2. A set of A-scans measured at neighboring locations in the physical object produces a cross-sectional image (tomogram or B-scan) of the physical object. A collection of individual B-scans collected at different transverse locations on the sample makes up a data volume or cube. Three-dimensional C-scans can be formed by combining a plurality of B-scans. For a particular volume of data, the term fast axis refers to the scan direction along a single B-scan whereas slow axis refers to the axis along which multiple B-scans are collected.

B-scans may be formed by any transverse scanning in the plane designated by the x-axis and y-axis. B-scans may be formed, for example, along the horizontal or x-axis direction, along the vertical or y-axis direction, along the diagonal of x-axis and y-axis directions, in a circular or spiral pattern, and combinations thereof. The majority of the examples discussed herein may refer to B-scans in the x-z axis directions but this disclosure may apply equally to any cross sectional image.

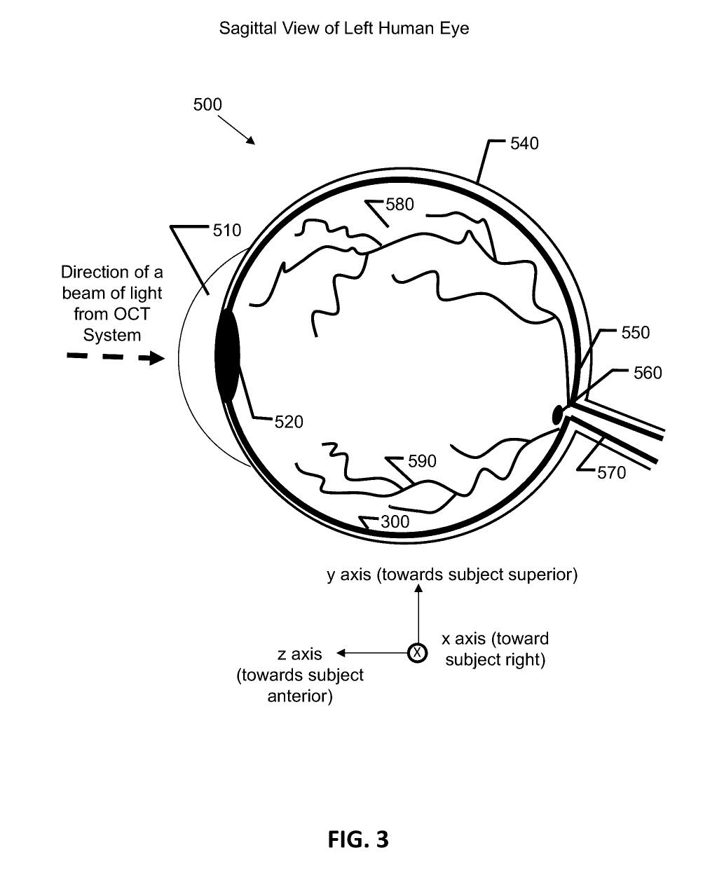

The physical object 210 may be any physical object. The physical object 210 may be a human eye, 500, as shown in a simplified manner in FIG. 3. The human eye comprises a cornea 510, a pupil 520, a retina 300, a choroid 540, a fovea region 550, an optic disk 560, an optic nerve 570, a vitreous chamber 580, and retinal blood vessels 590.

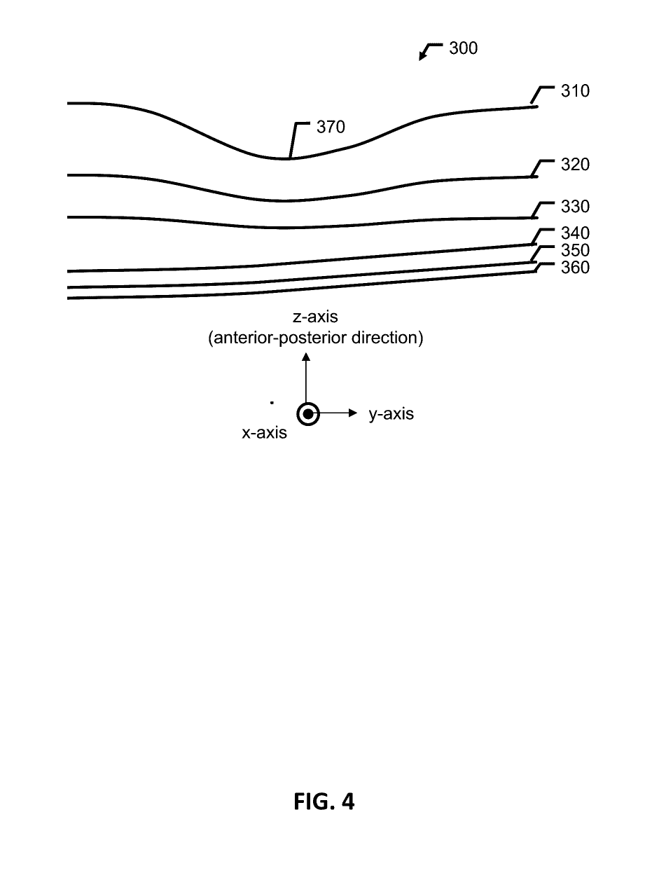

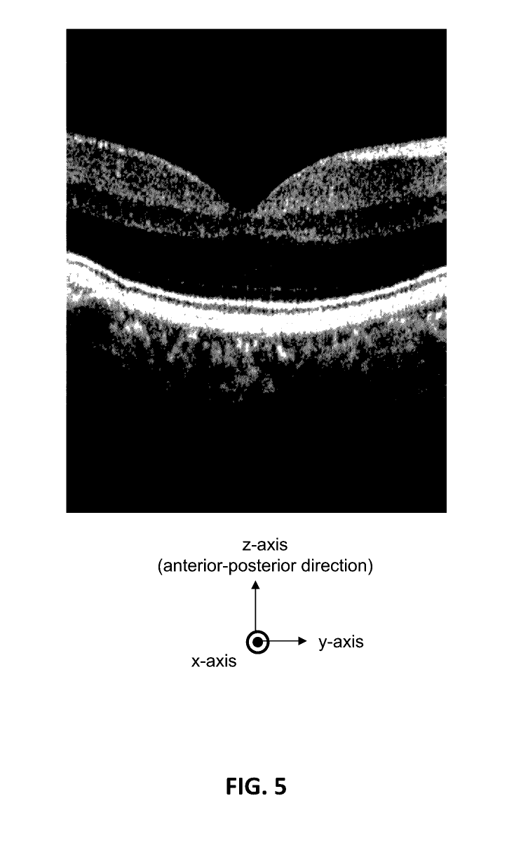

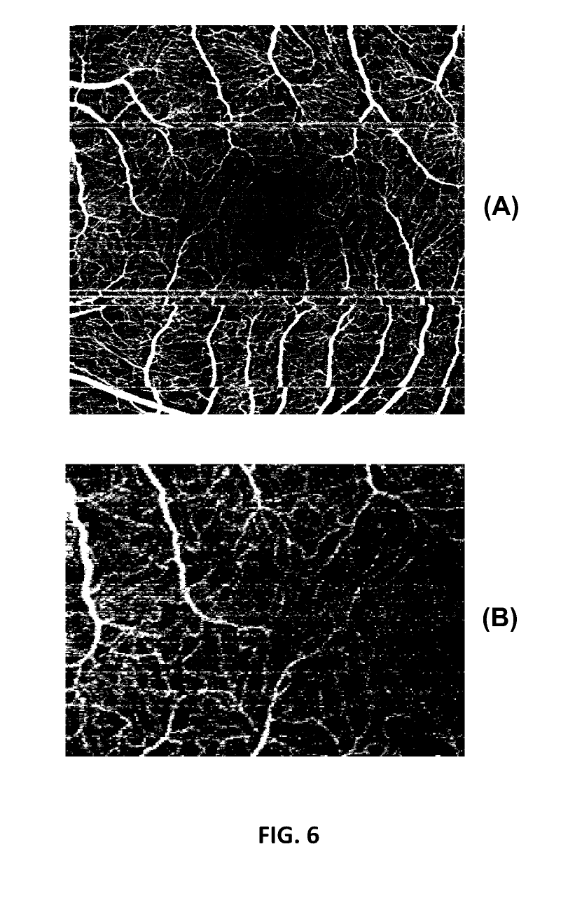

The physical object 210 may be tissue. An example of the tissue is a retina. A simplified cross-sectional image of layers of the retina 300 is schematically shown in FIG. 4. The retinal layers comprise a Nerve Fiber Layer (NFL) 310, External Limiting Membrane (ELM) 320, Inner/Outer Photoreceptor Segment 330, Outer Photoreceptor Segment 340, Retinal Pigment Epithelium (RPE) 350, Retinal Pigment Epithelium (RPE)/Bruch's Membrane Complex 360. FIG. 4 also schematically shows the fovea 370. FIG. 5 shows a cross-sectional OCT image of the fovea region of the retina. FIG. 6 shows (A) an exemplary en-face OCT angiography image of a retinal vasculature around optic disc, (B) a magnified region of the OCT image of (A).

The physical object may comprise any physical object as disclosed above. The physical object has a surface and a depth. For example, a fundus of an eye has an outer surface receiving light from outside environment through the pupil. The fundus of an eye also has a depth starting at and extending from its outer surface.

In this disclosure, a z-axis (“axial axis”) is an axis parallel to the beam of light extending into the depth of the physical object, the x-axis and the y-axis (“transverse axes”) are transverse, thereby perpendicular axes to the z-axis. Orientation of these three axes is shown in FIGS. 1-4, 7 and 9.

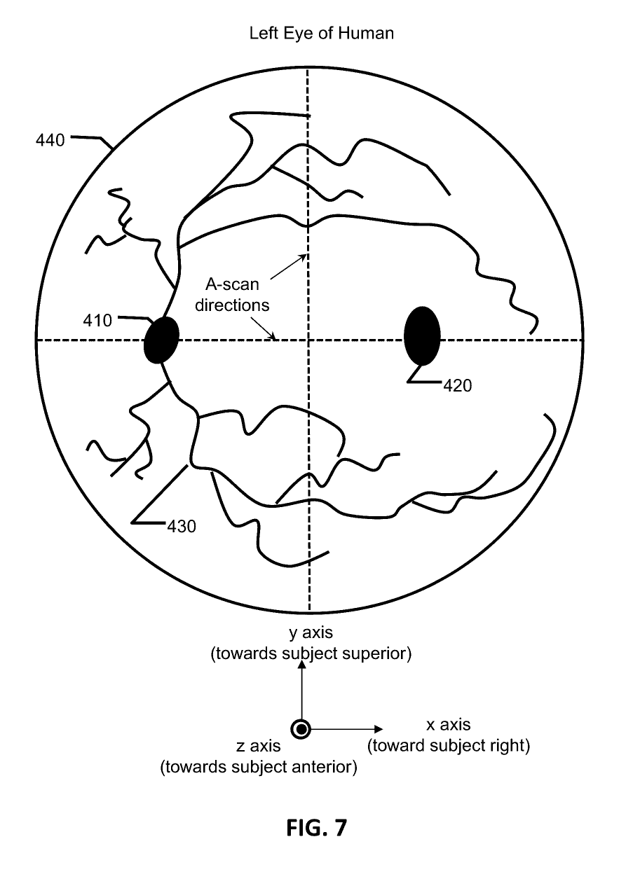

An example of the fundus of the eye is schematically shown in FIG. 7 in a simplified manner. In this circular visual field view 440 of the fundus of the eye, the anatomical landmarks are an optic disc 410, a fovea 420, and major blood vessels within the retina 430.

This disclosure relates to an optical coherence tomography (OCT) system for health characterization of an eye of a subject. The subject may be any mammal. The subject may be a human. This OCT system may have a configuration that (a) scans a tissue of the eye of a subject, which has a surface and a depth, with a beam of light that has a beam width and a direction; (b) acquires OCT signals from the scan; and (c) forms at least one B-scan cluster set using the acquired OCT signals.

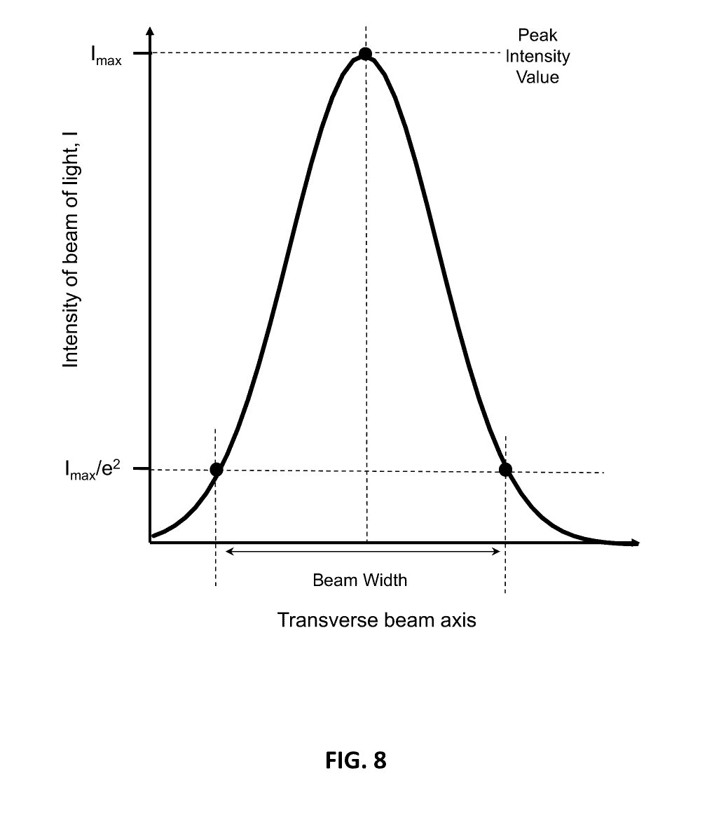

The beam of light provided by the OCT system has a width and an intensity at a location of the tissue of an eye. An example of the beam width is schematically shown in FIG. 8. This location may be at the surface of the tissue or within the tissue. In one example, at this location of the tissue, the beam of light may be focused (“focused beam of light”). For example, at this location the width of the beam of light may be at its smallest value. Cross-sectional area of the light beam may have any shape. For example, the cross-sectional area may have circular shape or elliptic shape. The intensity of the focused beam of light varies along its transverse axis, which is perpendicular to its propagation axis. This transverse beam axis may be a radial axis. The light beam intensity at the center of the light beam is at its peak value, i.e. the beam intensity is at its maximum, and decreases along its transverse axis, forming an intensity distribution. This distribution may be approximated by a Gaussian function, as shown in FIG. 8. The width of the beam of light (“beam width”) is defined as a length of line that intersects the intensity distribution at two opposite points at which the intensity is 1/e² times of its peak value. The light beam may comprise more than one peak. The peak with highest beam intensity is used to calculate the beam width. The beam width may be the focused beam of light. A typical beam width of a typical OCT system may vary in the range of 10 micrometers to 30 micrometers at the tissue location.

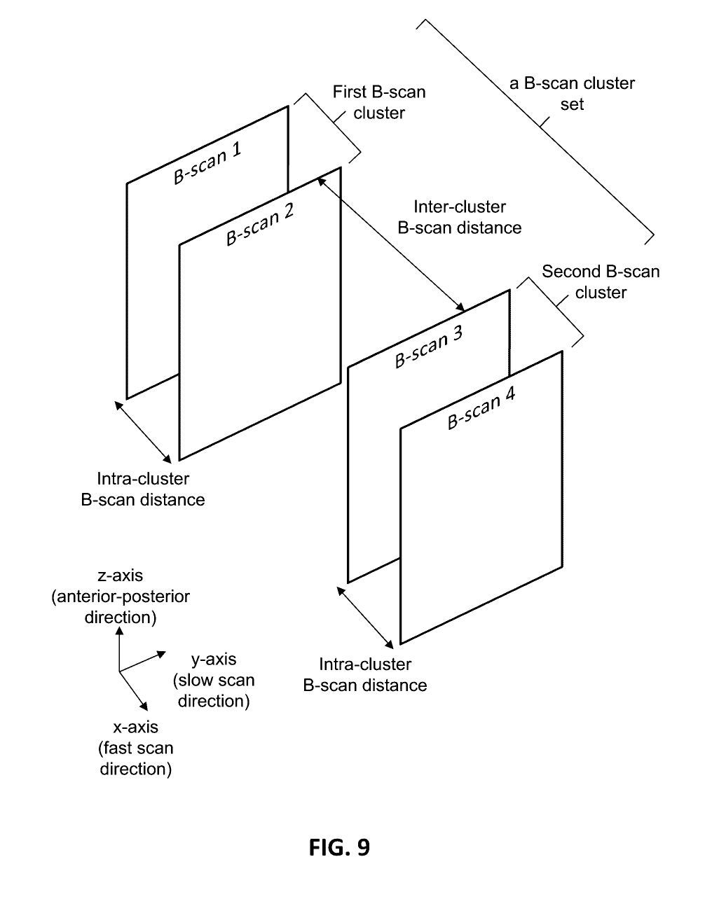

Each B-scan cluster set may include at least two B-scan clusters. Each B-scan cluster may include at least two B-scans. Each B-scan may include at least two A-scans. Each B-scan cluster set may be parallel to one another and parallel to the direction of the beam of light. The B-scans within each B-scan cluster set may be parallel to one another and parallel to the direction of the beam of light. An example of this system, shown in FIG. 9, comprises one B-scan cluster set comprising two B-scan clusters. And each B-scan cluster comprises two B-scans.

The OCT system for health characterization of an eye may have a configuration to form more than one B-scan cluster set. That is, a number of B-scan cluster set, P may be equal to or larger than 1, wherein P is an integer. For example, P may be 1, 2, 3, 4, 5, 10, 100, 1,000, 10,000, or 100,000.

Each B-scan cluster set may comprise any number of B-scan clusters, N equal to or greater than 2, wherein N is an integer. For example, N may be 2, 3, 4, 5, 10, 100, 1,000, 10,000, or 100,000.

Each B-scan cluster may comprise any number of B-scans, M equal to or greater than 2, wherein M is an integer. For example, M may be 2, 3, 4, 5, 10, 20, 100, 1,000, 10,000, or 100,000.

Each B-scan may comprise any number of A-scans, Q equal to or greater than 2, wherein M is an integer. For example, M may be 2, 3, 4, 5, 10, 20, 100, 1,000, 10,000, or 100,000.

Each A-scan, each B-scan, each B-scan cluster, and each B-scan cluster set may be acquired over a period of time. That is each A-scan, each B-scan, each B-scan cluster, and each B-scan cluster set may be formed at a different time than all other A-scans, B-scans, B-scan clusters, and B-scan cluster sets, respectively. In this disclosure, “first formed” means first formed in time; “next formed” means next formed in time; and “last formed” means last formed in time.

Each A-scan may be separated from any next A-scan by a distance (“A-scan distance”). The A-scan distance may be 0, at least 1 micrometer, or at least 10 micrometers.

Each B-scan within each B-scan cluster may be separated from any next formed B-scan within that B-scan cluster by a distance (“intra-cluster distance”) in the range of 0 to half of the beam width. For example, the intra-cluster distance may vary in the range of 0 to 15 micrometers.

The last formed B-scan within each B-scan cluster may be separated from the first formed B-scan within any next formed B-scan cluster (“inter-cluster distance”) by at least one micrometer. For example, the intra-cluster distance may vary in the range of 1 micrometer to 10 micrometers, 1 micrometer to 100 micrometers, or 1 micrometer to 1,000 micrometers.

The OCT system for health characterization of an eye may have a configuration to calculate an OCT angiography data using the at least one B-scan cluster and motion occurring within the eye tissue. The OCT angiography data may be calculated by using variations of intensity and/or phase of the OCT signals. This calculation may provide contrast. These variations may be variations caused by flow, speckle, and/or decorrelation of the OCT signal caused by eye tissue motion and/or flow in blood vessels of the eye tissue.

This OCT system may have a configuration that measures a feature of a vasculature within the eye tissue by using the calculated angiography data. The feature of a vasculature may be any feature of the vasculature. For example, the feature of a vasculature may be a size of a blood vessel, a spatial distance between each blood vessel, a cross-sectional area of a blood vessel, number of blood vessels, a shape of a blood vessel, a volume of blood vessels, and/or a spatial location of a blood vessel within the tissue. The size of a blood vessel may be any size of the blood vessel. For example, the size of a blood vessel may be its characteristic length, characteristic cross-sectional length, characteristic cross-sectional diameter, cross-sectional perimeter, or cross sectional circumference. The volume of blood vessels may be any volume of blood vessels. For example, the volume of blood vessels may be a total volume of blood vessels or a volume of blood vessels that have a specific blood vessel size.

The B-scan may yield a cross-sectional image of a tissue. For example, the B-scan shown in FIG. 5 is a cross-sectional OCT image of a retina. Such cross-sectional images may yield cross-sectional images of the vasculature in the eye tissue by using the calculated OCT angiography data. The features appearing on such cross-sectional images may be measured as features of the vasculature.

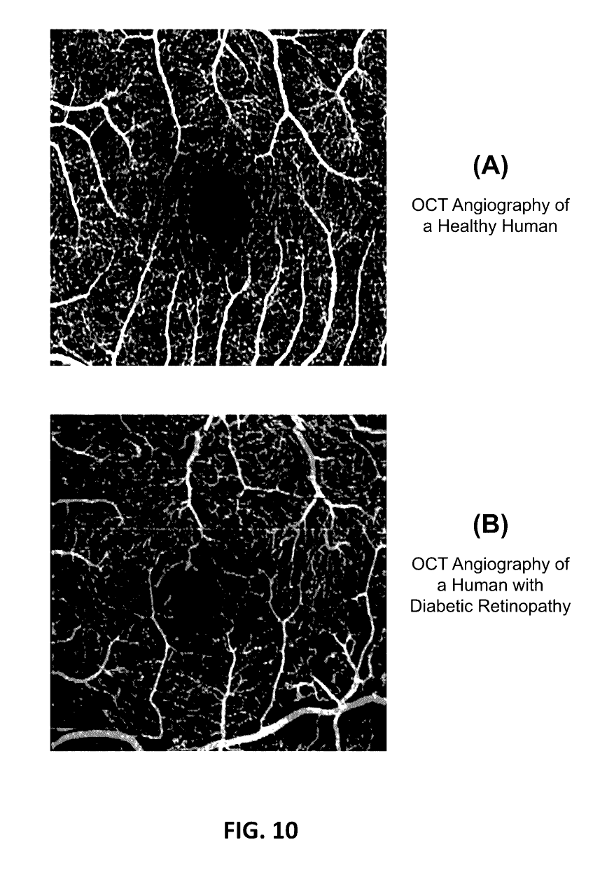

The B-scans may also yield en-face images of a vasculature in the eye tissue by using the calculated OCT angiography data, as shown in FIG. 10 by way of example. The features appearing on such en-face images may be measured as features of the vasculature.

The measured feature of the vasculature may be used to calculate a blood vessel population. The blood vessel population may be any blood vessel population. For example, the blood vessel population may be (a) a size distribution of blood vessels, (b) a spatial distance distribution of the blood vessels; (c) a cross-sectional area distribution of blood vessels; (d) a spatial location distribution of blood vessels; (e) a number of blood vessels per cross-sectional unit area of the tissue, and/or volume of the tissue; (f) a total of cross-sectional area of blood vessels per unit area of the tissue; (g) a volume of blood vessels per unit volume of the tissue; or (h) combinations thereof. The calculated blood vessel population may be a number and/or a histogram.

The blood vessel population may be used to identify a vascular anomaly and a spatial location of the vascular anomaly within the eye tissue of the subject. The vascular anomaly may be any vascular anomaly. For example, the vascular anomaly may be formation of new blood vessels, disappearance of blood vessels normally should have been present in a healthy tissue, enlargement of blood vessels, or contraction of blood vessels.

For this identification, for example, the subject's blood vessel population may be compared with that of a healthy eye tissue. The healthy eye tissue may be a tissue of the other eye of the subject. The healthy eye tissue may be a tissue from another region of the same eye of the subject. The healthy eye tissue may be a tissue of an eye of another (e.g. healthy) subject. The blood vessel population of a healthy subject may be determined by acquiring OCT angiography signals from the healthy subject. A database of healthy objects' blood vessel population may be formed and used for this comparison purposes. The vascular anomaly and its spatial location within the subject's eye tissue may thereby be determined by comparing such blood vessel population calculations.

For this identification, for example, the subject's blood vessel population information may be calculated more than once over a period of time. The vascular anomaly and its spatial location within the subject's eye tissue may be determined by comparing the blood vessel information calculated at a later time with that of an earlier time.

The OCT system may further have a configuration that acquires OCT signals to form at least two B-scan cluster sets.

The spatial distance between last formed B-scan of any B-scan cluster of each B-scan cluster set and first formed B-scan of any B-scan cluster of next formed B-scan cluster set (“inter-cluster-set distance”) may be equal to or greater than 1 micrometer. For example, the inter-cluster-set distance may be 20 micrometers.

In one example, the spatial distance between the last formed B-scan of any B-scan cluster of each B-scan cluster set and the first formed B-scan of the next formed B-scan cluster set (“inter-cluster-set distance”) may be equal to or greater than 1 micrometer. For example, the inter-cluster-set distance may be 20 micrometers.

In another example, the spatial distance between the last formed B-scan of first B-scan cluster of each B-scan cluster set and the first formed B-scan of the next formed B-scan cluster set (“inter-cluster-set distance”) may be equal to or greater than 1 micrometer. For example, the inter-cluster-set distance may be 20 micrometers.

Yet, in another example, the spatial distance between the last formed B-scan of each B-scan cluster set and the first formed B-scan of the next formed B-scan cluster set (“inter-cluster-set distance”) may be equal to or greater than 1 micrometer. For example, the inter-cluster-set distance may be 20 micrometers.

The OCT system for health characterization of an eye may also have a configuration that calculates a first OCT angiography data and a second OCT angiography data. The first OCT angiography data may be calculated at a first inter-cluster-set distance. And the second OCT angiography data may be calculated at a second inter-cluster-set distance. The second inter-cluster-set distance may be smaller than the first inter-cluster-set distance. The first inter-cluster-set distance may be at least 4 micrometers, at least 10 micrometers, at least 20 micrometers, or at least 100 micrometers. The second inter-cluster-set distance may be at least 1 micrometer, at least 2 micrometers, at least 5 micrometers, or at least 10 micrometers.

The first OCT angiography data may be used in identification of a vascular anomaly and a spatial location of the vascular anomaly within the eye tissue by using the blood vessel population. The second OCT angiography data may be calculated by scanning a tissue at or around the vascular anomaly and acquiring OCT signals from this scan. Since the second OCT angiography data is calculated at a smaller inter-cluster-set distance, it may yield more detailed information related to the anomaly.

The identified vascular anomaly and its spatial location may be used in determination of a health disorder. Examples of such disorders are retinal detachments or tears, macular degeneration, diabetic retinopathy (DR), retinovascular occlusions, retinal cancers, retinal abnormalities, presence of drusen or exudates, retinal edema, and the like.

FIG. 10 shows (A) an exemplary en-face OCT angiography image of retinal vasculature around optic disc of a healthy human, (B) an exemplary en-face OCT angiography image of retinal vasculature around optic disc of a human with diabetic retinopathy. The healthy human's eye has more blood vessels and distances between the blood vessels are smaller as compared to those of the human with diabetic retinopathy. Thus, the human with diabetic retinopathy lost some of his/her blood vessels.

The eye health characterization method by OCT may also comprise high resolution OCT scans. Examples of such high resolution OCT scans are:

(a) En face images of high resolution volumetric OCT data sets. These en face images can be based on depth projections of individual segmented retinal layers, or based off the entire retinal depth

(b) Cross-sectional OCT B-scans calculated for selected regions across the retina. Depending on how sparsely sampled these scans are, this method is an efficient way of achieving wide field screening capabilities in a short amount of imaging time.

(c) A series of high resolution OCT volumetric scans, which only extend for a small region in the slow scanning axis direction. Using the OCT cross-sectional images, projections and analysis along the slow axis can allow for more direct visualization of the microvascular features for analysis and quantification.

Medical diagnostics typically rely on quantitative metrics to evaluate the health and status of a patient. With the development of microvascular visualization capabilities within the retina and choroid, this provides new opportunities to evaluate the health of the eye based on the microvasculature information.

This disclosure thereby also relates to a quantitative metric based on the retinal microvascular density acquired by the retinal health characterization method by OCT disclosed above. This metric may be used to characterize the health of the retina and evaluate the progression of diseases such as diabetic retinopathy.

### EXAMPLE 1

High resolution OCT volumetric scans may be acquired over the retina. En face images from several different retinal layers may be extracted and the vasculature may be isolated from these layers, and the two dimensional vascular density maps may be calculated. These vascular density maps may spatially vary across the retina, typically going to zero in the foveal avascular zone. The vascular density maps from the retinal layers may be aggregated into a single quantitative number to characterize the vascular health of the retina.

### EXAMPLE 2

Wide field high resolution cross-sectional OCT angiography scans may be acquired at sparsely sampled locations across the retina. For example, for a 20×20 degree field of view, OCT angiography images may be acquired at 5° separations to allow for quicker, wide field acquisitions. Microvascular projections may be identified within the cross-sectional scans, and vascular densities may be calculated along the horizontal direction of the scan. These calculations may be repeated for the other cross-sectional images, giving a 2D vascular density map across a wide field of the retina, with low resolution along the slow axis direction.

### EXAMPLE 3

Wide field cross-sectional OCT angiography scans may be acquired, but the entire horizontal range may be broken up into several, smaller acquisitions which may reduce the phase noise errors present. These acquisitions may be stitched together in the horizontal direction to cover a wider range. Each of these acquisitions may also be acquired as a small volumetric scan in the slow scanning axis directly, only about 0.5° to provide some additional context and statistics to identify the microvasculature within the retina. These scans are also repeated sparsely over the slow axis direction to cover a wider field of view.

The OCT system disclosed above may be used for any OCT related application. For example, this system maybe used in forming larger field of view OCT images of the physical object. This system may be incorporated into methods and systems related to OCT based angiography. For example, the choroidal vasculature may be identified in more detail by using the OCT system disclosed above. The OCT system may also be used in diagnosis and/or treatment of health conditions such as diseases. For example, the OCT system may be used in characterization of retinal health.

The OCT system disclosed above may provide any information related to any physical object. For example, this system may provide 2D (i.e. cross-sectional) images, en-face images, 3-D images, metrics related to a health condition, and the like. This system may be used together with any other system. For example, the OCT system may be used together with an ultrasound device, or a surgical system for diagnostic or treatment purposes. The OCT system may be used to analyze any physical object. For example, the OCT system may be used in analysis, e.g. formation of images, of, for example, any type of life forms and inanimate objects. Examples of life forms may be animals, plants, cells or the like.

Unless otherwise indicated, the processing unit 140 that has been discussed herein may be implemented with a computer system configured to perform the functions that have been described herein for this unit. The computer system includes one or more processors, tangible memories (e.g., random access memories (RAMs), read-only memories (ROMs), and/or programmable read only memories (PROMS)), tangible storage devices (e.g., hard disk drives, CD/DVD drives, and/or flash memories), system buses, video processing components, network communication components, input/output ports, and/or user interface devices (e.g., keyboards, pointing devices, displays, microphones, sound reproduction systems, and/or touch screens).

The computer system for the processing unit 140 may include one or more computers at the same or different locations. When at different locations, the computers may be configured to communicate with one another through a wired and/or wireless network communication system.

The computer system may include software (e.g., one or more operating systems, device drivers, application programs, and/or communication programs). When software is included, the software includes programming instructions and may include associated data and libraries. When included, the programming instructions are configured to implement one or more algorithms that implement one or more of the functions of the computer system, as recited herein. The description of each function that is performed by each computer system also constitutes a description of the algorithm(s) that performs that function.

The software may be stored on or in one or more non-transitory, tangible storage devices, such as one or more hard disk drives, CDs, DVDs, and/or flash memories. The software may be in source code and/or object code format. Associated data may be stored in any type of volatile and/or non-volatile memory. The software may be loaded into a non-transitory memory and executed by one or more processors.

Any combination of features, products, and methods disclosed above are within the scope of this disclosure.

Unless otherwise stated, all measurements, values, ratings, positions, magnitudes, sizes, and other specifications that are set forth in this disclosure are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

In this disclosure, the indefinite article “a” and phrases “one or more” and “at least one” are synonymous and mean “at least one”.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

1. An optical coherence tomography (OCT) system for health characterization of an eye having a configuration that:
(a) scans tissue of an eye of a subject, which has a surface and a depth, with a beam of light that has a beam width and a direction;
(b) acquires OCT signals from the scan;
(c) forms at least one B-scan cluster set using the acquired OCT signals such that:
each B-scan cluster set includes at least two B-scan clusters;
each B-scan cluster includes at least two B-scans;
each B-scan includes at least two A-scans;
if there is more than one B-scan cluster set, each B-scan cluster set is parallel to one another;
each B-scan cluster set is parallel to the direction of the beam of light;
the B-scans within each B-scan cluster set are parallel to one another and parallel to the direction of the beam of light;
each A-scan, each B-scan, each B-scan cluster, and each B-scan cluster set are formed at a different time than all other A-scans, B-scans, B-scan clusters, and B-scan cluster sets, respectively;
each A-scan is separated from any next formed A-scan by a distance (“A-scan distance”);
each B-scan within each B-scan cluster is separated from any next formed B-scan within that B-scan cluster by a distance (“intra-cluster distance”) in the range of 0 to half of the beam width; and
the last formed B-scan within each B-scan cluster is separated from the first formed B-scan within any next formed B-scan cluster (“inter-cluster distance”) by at least one micrometer;
(d) calculates OCT angiography data using the at least one B-scan cluster set formed at (c) of this claim and based on motion occurring within the eye tissue; and
(e) determines a feature of a vasculature within the eye tissue by using the calculated angiography data.

2. The system of claim 1, further having a configuration such that the OCT angiography data is calculated by using variations of intensity and/or phase of the OCT signals to provide contrast.

3. The system of claim 2, further having a configuration such that the variations are the variations caused by flow, speckle, or decorrelation of an OCT signal within the OCT signals that is caused by eye tissue motion or flow in blood vessels of the eye tissue.

4. The system of claim 1, further having a configuration such that the feature of a vasculature is a size of a blood vessel, a spatial distance between blood vessels, a cross-sectional area of a blood vessel, number of blood vessels, a shape of a blood vessel, a volume of a blood vessel, or a spatial location of a blood vessel within the tissue.

5. The system of claim 4, further having a configuration that calculates a blood vessel population using the determined feature of the vasculature.

6. The system of claim 5, further having a configuration such that the blood vessel population is:
a size distribution of the blood vessels;
a spatial distance distribution of the blood vessels;
a cross-sectional area distribution of the blood vessels;
a spatial location distribution of the blood vessels;
a number of the blood vessels per cross-sectional unit area of the tissue, or per volume of the tissue;
a volume of the blood vessels per volume of the tissue;
a total cross-sectional area of the blood vessels per unit area of the tissue; or
a combination thereof.

7. The system of claim 6, further having a configuration that identifies a vascular anomaly and a spatial location of the vascular anomaly within the eye tissue by using the calculated blood vessel population.

8. The system of claim 6, further having a configuration that identifies a vascular anomaly and a spatial location of the vascular anomaly within the eye tissue by comparing the calculated blood vessel population with that of a healthy eye tissue.

9. The system of claim 6, further having a configuration that identifies a vascular anomaly and a spatial location of the vascular anomaly within the eye tissue by calculating the blood vessel population of the subject at different times and comparing the blood vessel population calculated at a later time with that of an earlier time.

10. The system of claim 7, further having a configuration that:
(a) forms at least two B-scan cluster sets of the type recited in claim 1 such that last formed B-scan of one of the B-scan clusters of each B-scan cluster set is separated from first formed B-scan of one of the B-scan clusters of the next formed B-scan cluster set (“inter-cluster-set distance”) by a first distance;
(b) calculates a first OCT angiography data using the at least two B-scan cluster sets formed at (a) of this claim and a motion occurring within the eye tissue;
(c) identifies a vascular anomaly and a spatial location of the vascular anomaly within the eye tissue by using the blood vessel population;
(d) repeats (a) of this claim at a second inter-cluster-set distance at or around the spatial locations of the identified vascular anomaly such that the second inter-cluster-set distance is smaller than the first inter-cluster-set distance;
(e) calculates a second OCT angiography data using the at least two B-scan cluster sets formed at (d) of this claim and motion occurring within the tissue; and
(f) determines a feature of blood vessels within the eye tissue by using the second OCT angiography data.

11. The system of claim 10, further having a configuration that identifies a vascular anomaly and a spatial location of the vascular anomaly within the eye tissue by comparing the blood vessel population information with that of a healthy eye tissue.

12. The system of claim 10, further having a configuration that identifies a vascular anomaly and a spatial location of the vascular anomaly within the eye tissue by calculating the blood vessel population information of the subject at different times and comparing the blood vessel population information calculated at a later time with that of an earlier time.

13. The system of claim 10, further having a configuration such that the A-scan distance of the second angiography data is smaller than the A-scan distance of the first angiography data.

14. Non-transitory, tangible, computer-readable storage media containing a program of instructions that causes an optical coherence tomography (OCT) system for health characterization of an eye running the program of instructions to:
(a) scans tissue of an eye of a subject, which has a surface and a depth, with a beam of light that has a beam width and a direction;
(b) acquires OCT signals from the scan;
(c) forms at least one B-scan cluster set using the acquired OCT signals such that:
each B-scan cluster set includes at least two B-scan clusters;
each B-scan cluster includes at least two B-scans;
each B-scan includes at least two A-scans;
if there is more than one B-scan cluster set, each B-scan cluster set is parallel to one another;
each B-scan cluster set is parallel to the direction of the beam of light;
the B-scans within each B-scan cluster set are parallel to one another and parallel to the direction of the beam of light;
each A-scan, each B-scan, each B-scan cluster, and each B-scan cluster set are formed at a different time than all other A-scans, B-scans, B-scan clusters, and B-scan cluster sets, respectively;
each A-scan is separated from any next formed A-scan by a distance (“A-scan distance”);
each B-scan within each B-scan cluster is separated from any next formed B-scan within that B-scan cluster by a distance (“intra-cluster distance”) in the range of 0 to half of the beam width; and
the last formed B-scan within each B-scan cluster is separated from the first formed B-scan within any next formed B-scan cluster (“inter-cluster distance”) by at least one micrometer;
(d) calculates OCT angiography data using the at least one B-scan cluster set formed at (c) of this claim and based on motion occurring within the eye tissue; and
(e) determines a feature of a vasculature within the eye tissue by using the calculated angiography data.

15. The storage media of claim 14, further having a configuration that calculates a blood vessel population using the determined feature of the vasculature.

16. The storage media of claim 15, further having a configuration such that the blood vessel population is:
a size distribution of the blood vessels;
a spatial distance distribution of the blood vessels;
a cross-sectional area distribution of the blood vessels;
a spatial location distribution of the blood vessels;
a number of the blood vessels per cross-sectional unit area of the tissue, or per volume of the tissue;
a volume of the blood vessels per volume of the tissue;
a total cross-sectional area of the blood vessels per unit area of the tissue; or
a combination thereof.

17. The storage media of claim 16, further having a configuration that identifies a vascular anomaly and a spatial location of the vascular anomaly within the eye tissue by using the calculated blood vessel population.

18. The storage media of claim 17, further having a configuration that:
(a) forms at least two B-scan cluster sets of the type recited in claim 14 such that last formed B-scan of one of the B-scan clusters of each B-scan cluster set is separated from first formed B-scan of one of the B-scan clusters of the next formed B-scan cluster set (“inter-cluster-set distance”) by a first distance;
(b) calculates a first OCT angiography data using the at least two B-scan cluster sets formed at (a) of this claim and a motion occurring within the eye tissue;
(c) identifies a vascular anomaly and a spatial location of the vascular anomaly within the eye tissue by using the blood vessel population;
(d) repeats (a) of this claim at a second inter-cluster-set distance at or around the spatial locations of the identified vascular anomaly such that the second inter-cluster-set distance is smaller than the first inter-cluster-set distance;
(e) calculates a second OCT angiography data using the at least two B-scan cluster sets formed at (d) of this claim and motion occurring within the tissue; and
(f) determines a feature of blood vessels within the eye tissue by using the second OCT angiography data.

19. The storage media of claim 18 wherein the program of instructions causes the computer system running the program of instructions to identify a vascular anomaly and a spatial location of the vascular anomaly within the eye tissue by comparing the blood vessel population information with that of a healthy eye tissue.

20. The storage media of claim 18 wherein the program of instructions causes the computer system running the program of instructions to identify a vascular anomaly and a spatial location of the vascular anomaly within the eye tissue by calculating the blood vessel population information of the subject at different times and comparing the blood vessel population information calculated at a later time with that of an earlier time.
